(19) United States
(12) Patent Application Publication
(10) Pub. No.: US 2019/0244716 A1
(43) Pub. Date: Aug. 8, 2019

(54) AUTOMATIC BIOPSY REPORTING SYSTEMS AND METHODS

(71) Applicant: KONINKLIJKE PHILIPS N.V., EINDHOVEN (NL)

(72) Inventors: Lucas de Melo OLIVEIRA, Wilmington, MA (US); Gabriel Ryan MANKOVICH, Boston, MA (US); Amir Mohammad TAHMASEBI MARAGHOOSH, Arlington, MA (US)

(21) Appl. No.: 16/333,278

(22) PCT Filed: Oct. 13, 2017
(86) PCT No.: PCT/EP2017/076143

§ 371 Date: Mar. 14, 2019

Related U.S. Application Data
(60) Provisional application No. 62/408,961, filed on Oct. 17, 2016.

(51) Int. Cl. G16H 70/60; G16H 15/00

(52) U.S. Cl. CPC G16H 70/60; G16H 15/00

(57) ABSTRACT

A biopsy reporting system (101) employs a biopsy procedure database (11) and a biopsy reporting controller (102). In operation, the biopsy procedure database (11) stores a biopsy procedure registration and a pathology report. The biopsy procedure registration includes sample tissue data record informative of biopsy extractions of a plurality of biopsy samples from a patient anatomy. The pathology report includes pathological data informative of a pathological diagnosis of each biopsy sample of the plurality of biopsy samples. Responsive to a storage of the biopsy procedure registration and the pathology report within the biopsy procedure database (11), the biopsy reporting controller (102) automatically links the pathology report to the biopsy procedure registration. Responsive to the automatic linking of the pathology report to the biopsy procedure registration, the biopsy reporting controller (102) automatically links a pathological diagnosis of each biopsy sample to a corresponding biopsy extraction of each biopsy sample.

### FIELD OF THE INVENTION

The present disclosure generally relates to biopsy reporting systems and methods.

The present disclosure specifically relates to improving a workflow between an extraction of biopsy samples and a pathological diagnostic review of the extracted biopsy samples for both targeted and systematic biopsy procedures (i.e., a biopsy workflow).

### BACKGROUND OF THE INVENTION

With an introduction of image fusion systems for interventional biopsy procedures (e.g., a prostate biopsy), it is now feasible to acquire targeted biopsies in lieu of acquiring conventional systematic biopsies. During a typical targeted prostate biopsy, a urologist reviews target biopsy locations that are pre-determined by a radiologist. At the time of biopsy, a nurse reads out loud the biopsy locations and urologist guides the biopsy needle to the desired location to acquire the target biopsy. The specimen is then dispensed into a container with the same label as the biopsy location. Besides targeted biopsies, the urologist may acquire systematic biopsies. Following the same procedure, samples are taken and left in containers with labels indicating the systematic location of the biopsy. Such labels could be based on a schematic anatomy region nomenclature. For example in a prostate biopsy, LFL and RMM labels are assigned for Left Far Lateral and Right Mid Medial regions, respectively. Finally, all biopsy samples are sent to a pathology department. A pathologist analyses each sample tissue and prepares a pathology report containing the diagnosis for each biopsy sample by examining cells and tissues under a microscope. The pathology report is thereafter stored within a biopsy procedure database for review by the biopsy staff.

There are two main problems in the aforementioned workflow between the extraction of biopsy samples and the pathological diagnostic review of the extracted biopsy samples (i.e., a biopsy workflow) that impact productive, efficiency and more importantly the final treatment plan for a given patient.

The first problem is that there is no link in the biopsy procedure database between a registered biopsy procedure involving an extraction of the biopsy samples for a particular patient and a pathology report of the extracted biopsy samples for that patient. Consequently, physicians have to manually browse all the pathology reports stored within the the biopsy procedure database to find the particular pathology report that describes the diagnosis for the registered biopsy procedure of the biopsy sample extractions for that patient. This manual browsing is disruptive to the biopsy workflow, particularly due to a difficulty and time consumption in manually associating a pathology report to the correct registered biopsy procedure.

For example, as shown in FIG. 1A, a biopsy procedure system 10 (e.g., UroNav® Fusion Biopsy System) is operated in accordance with a target biopsy procedure and/or a systematic biopsy procedure to generate and store biopsy procedure registrations (“BPR”) 12 for a plurality of biopsy patients within a biopsy procedure database 11. Each biopsy procedure registration 12 is informative of each biopsy sample tissue extraction of a particular patient (i.e., sample tissue data record) with each sample tissue extraction being tagged with a biopsy sample label. Each biopsy sample (“BS”) is stored within a container 13 and provided to a pathology report system 20 (e.g., Pacs™ Solution) whereby each container 13 is tagged with a sample location label, which typically differs from the corresponding biopsy sample label stored within the biopsy procedure database 11, particularly for a targeted biopsy procedure.

Subsequent to a tagging of containers 13, a pathological diagnosis of all biopsy samples are performed and the pathology report system 20 is operated to generate pathology reports (“PR”) 21, copies of which are transmitted to biopsy procedure system 10 and stored within the biopsy procedure database 11. Each pathology report 21 is informative of a pathological diagnosis of each biopsy sample of a particular patient (i.e., sample diagnostic data entry) whereby each biopsy sample diagnosis is tagged with a pathology location label. Biopsy procedure system 10 fails to provide a mechanism, as symbolically shown, for linking a biopsy procedure registration 12 and a corresponding pathology report 21 as stored within biopsy procedure database 11. As a result, to identify a pathology report 21 for a particular patient, a biopsy staffer has to manually browse all the pathology reports 21 stored within the biopsy procedure database 11 to find a particular pathology report 21 corresponding to the biopsy procedure registration 12 for that patient. This disrupts the biopsy workflow.

The second problem of a biopsy workflow is, after a biopsy staffer has found the particular pathology report corresponding to the subject biopsy procedure registration, there is no link between the sample tissue data record registered in the biopsy procedure registration and associated sample diagnostic data entry reported in the pathology report. More particularly, a pathology location label is assigned to a particular biopsy sample in the pathology report to tag the sample diagnostic data entry for the particular biopsy sample, but the biopsy procedure system typically will have a different biopsy sample label automatically generated for the same sample tissue. As a result, biopsy staffers have to spend time to manually link each sample diagnostic data entry from the pathology report with a corresponding sample tissue data record of a biopsy procedure registration. This is counterproductive and increase the chance to introduce human errors in the diagnostic process.

For example, as shown in FIG. 1B, a biopsy procedure registration 12(1) of an extraction of four (4) biopsy samples for a particular patient is generated with a listing of sample tissue data record 15 for each biopsy sample with each sample tissue data record 15 being tagged with a biopsy sample label 14. Conversely, a pathology report 21(1) for the same patient listing sample diagnostic data entry 23 for each diagnosed biopsy sample with each sample diagnostic data entry 23 being tagged by a pathology location label 22. While sample location labels (not shown) of biopsy samples within containers 13 and the pathology location labels 22 have a similar basis in identifying a biopsy sample location, biopsy procedure system 10 fails to provide any mechanism, as symbolically shown, for linking each sample diagnostic data entry 23 to a corresponding sample tissue data record 15. As a result, after a biopsy staffer has determined that biopsy procedure registration 12(1) corresponds to particular pathology report 21(1), then the biopsy staffer has to determine which sample diagnostic data entry 23 corresponds to a particular sample tissue data record 15, such as for, example, a sample diagnostic data entry 23(3) that corresponds to sample tissue data record 15(1). This further disrupts the biopsy workflow.

### SUMMARY OF THE INVENTION

The present disclosure provides systems, controllers and methods improving existing biopsy procedure systems, controller and methods by (1) automatically linking a pathology report to a corresponding biopsy procedure registration as stored within a biopsy procedure database of a biopsy procedure system, and (2) automatically linking each sample diagnostic data entry reported in the linked pathology report to a corresponding sample tissue data record registered in the linked biopsy procedure registration stored as stored within the biopsy procedure database of the biopsy procedure system.

As a result, for purposes of reviewing a pathological diagnosis of the biopsy, a biopsy staffer retrieving the biopsy procedure registration from the biopsy procedure database will be automatically provided with a link (e.g., a hyperlink) to the corresponding pathology report whereby the biopsy staffer may further retrieve the pathology report from the biopsy procedure database to thereby review each linked sample tissue data record and same diagnostic information. Conversely, the biopsy staffer may retrieve the pathology report and automatically be provided with a link (e.g., a hyperlink) to the corresponding biopsy procedure registration whereby the biopsy staffer may further retrieve the biopsy procedure registration to thereby review each linked sample tissue data record and same diagnostic information.

One embodiment of the inventions of the present disclosure is a biopsy reporting system employing a biopsy procedure database and a biopsy reporting controller. In operation, the biopsy procedure database stores a biopsy procedure registration and a pathology report. The biopsy procedure registration includes sample tissue data informative of biopsy extractions of a plurality of biopsy samples from a patient anatomy. The pathology report includes sample diagnostic data informative of a pathological diagnosis of each biopsy sample of the plurality of biopsy samples. Responsive to a storage of the biopsy procedure registration and the pathology report within the biopsy procedure database, the biopsy reporting controller automatically links the pathology report to the biopsy procedure registration. Responsive to the automatic linking of the pathology report to the biopsy procedure registration, the biopsy reporting controller automatically links one of the pathological diagnoses of biopsy sample to a corresponding biopsy extraction of each biopsy sample.

In a second embodiment of the inventions of the present disclosure, the biopsy reporting controller employs a pathology report linker, a pathological location label identifier, a biopsy sample label mapper and a biopsy sample label linker.

In operation, the pathology report linker automatically links a pathology report stored within a database to a corresponding biopsy procedure registration among a plurality of biopsy procedure registrations stored within the biopsy procedure database.

The pathology location label identifier identifies pathology location labels within the pathology report responsive to a linkage by the pathology report linker of the pathology report to the corresponding biopsy procedure registration.

The biopsy sample label mapper maps a plurality of biopsy sample labels of the biopsy procedure registration to an anatomical schematic of a patient anatomy.

The biopsy sample label linker automatically links each biology sample label among the plurality of biology sample labels to a corresponding pathology location label among the plurality of pathology location responsive to an identification by the pathology location label identifier of the pathology location labels within the pathology report and further responsive to a mapping by the biopsy sample label mapper of the biopsy sample labels to the anatomical schematic.

For purposes of describing and claiming the inventions of the present disclosure:

(1) the term “biopsy procedure” broadly encompasses all biopsy procedures as known in the art of the present disclosure and hereinafter conceived. Examples of a biopsy procedures, but is not limited to, bone marrow biopsy, needle biopsy and endoscopic biopsy;

(2) the terms “biopsy procedure registration”, “sample tissue data record” “pathology report”, “sample diagnostic data entry”, “biopsy sample label”, “biopsy location label” and “pathological location label” are to interpreted as known in the art of the present disclosure and exemplary described herein for the implementation of a biopsy procedure;

(3) the term “biopsy procedure database” broadly encompasses all devices, as known in the art of the present disclosure and hereinafter conceived, structurally configured for the collection, organization and retrieval of any data related to an extraction of biopsy samples and a pathological diagnostic review of the extracted biopsy samples;

(4) the terms “link”, “linkage” and “linking” are to be interpreted as known in the art of the present disclosure and exemplary described herein as related to linking data collected and organized within a database whereby the linked data may be co-retrieved from the biopsy procedure database for display, printing, transmission, etc.

(5) the term “biopsy reporting method” broadly encompasses an incorporation of the inventive principles of the present disclosure within a biopsy procedure for automatically linking a pathology report to a corresponding biopsy procedure registration in a biopsy procedure system, and for automatically linking sample tissue data record registered in the biopsy procedure system with the corresponding sample diagnostic data entry reported in pathology report as exemplary described herein.

(6) the term “biopsy procedure system” broadly encompasses all biopsy procedures systems, as known in the art of the present disclosure and hereinafter conceived, for conducting a biopsy procedure. An example of a biopsy procedure system include, but is not limited to, a UroNav® Fusion Biopsy System;

(7) the term “biopsy reporting system” broadly encompasses an incorporation of the inventive principles of the present disclosure within a biopsy procedure system as exemplary described herein for automatically linking a pathology report to its respective biopsy procedure registration as stored within a biopsy procedure database, and for automatically linking sample tissue data record registered in the biopsy procedure database to corresponding sample diagnostic data entry reported in pathology report;

(8) the term “controller” broadly encompasses all structural configurations, as understood in the art of the present disclosure and as exemplary described herein, of an application specific main board or an application specific integrated circuit for controlling an application of various inventive principles of the present disclosure as subsequently described herein. The structural configuration of the controller may include, but is not limited to, processor(s), computer-usable/computer readable storage medium(s), an operating system, application module(s), peripheral device controller(s), slot(s) and port(s). A controller may be installed within or remotely accessible by a workstation. Examples of a “workstation” include, but are not limited to, an assembly of one or more computing devices, a display/monitor, and one or more input devices (e.g., a keyboard, joysticks and mouse) in the form of a standalone computing system, a client computer of a server system, a desktop or a tablet.

(9) the term “module” broadly encompasses a module incorporated within or accessible by a controller consisting of an electronic circuit and/or an executable program (e.g., executable software stored on non-transitory computer readable medium(s) and/or firmware) for executing a specific application; and

(10) the terms “data” broadly encompasses all forms of a detectable physical quantity or impulse (e.g., voltage, current, or magnetic field strength) as understood in the art of the present disclosure and as exemplary described herein for transmitting information and/or instructions in support of applying various inventive principles of the present disclosure as subsequently described herein. Data communication encompassed by the inventions of the present disclosure may involve any communication method as known in the art of the present disclosure including, but not limited to, data transmission/reception over any type of wired or wireless datalink and a reading of data uploaded to a computer-usable/computer readable storage medium; and

The foregoing embodiments and other embodiments of the inventions of the present disclosure as well as various features and advantages of the present disclosure will become further apparent from the following detailed description of various embodiments of the inventions of the present disclosure read in conjunction with the accompanying drawings. The detailed description and drawings are merely illustrative of the inventions of the present disclosure rather than limiting, the scope of the inventions of present disclosure being defined by the appended claims and equivalents thereof.

### BRIEF DESCRIPTION OF THE DRAWINGS

FIG. 1A illustrates a block diagram of an exemplary embodiment of a biopsy procedure system providing a non-linkage between a biopsy procedure registration and a pathology report as known in the art of the present disclosure.

FIG. 1B illustrates a block diagram of an exemplary non-linkage of a biopsy procedure registration and a pathology report as known in the art of the present disclosure.

FIG. 2A illustrates a block diagram of an exemplary embodiment of a biopsy procedure system providing an automatic linkage between a biopsy procedure registration and a pathology report in accordance with the inventive principles of the present disclosure.

FIG. 2B illustrates a block diagram of an exemplary automatic linkage of a biopsy procedure registration and a pathology report in accordance with the inventive principles of the present disclosure.

FIG. 3A illustrates a block diagram of an exemplary embodiment of the biopsy reporting system of FIG. 2A in accordance with the inventive principles of the present disclosure.

FIG. 3B illustrates a block diagram of an exemplary first embodiment of the biopsy reporting system of FIG. 3A in accordance with the inventive principles of the present disclosure.

FIG. 3C illustrates a block diagram of an exemplary second embodiment of the biopsy reporting system of FIG. 3A in accordance with the inventive principles of the present disclosure.

FIG. 4 illustrates a procedural flow of an exemplary embodiment of a biopsy reporting method in accordance with the inventive principles of the present disclosure.

FIG. 5 illustrates a block diagram of an exemplary embodiment of a biopsy reporting controller of FIGS. 3A-3C in accordance with the inventive principles of the present disclosure.

FIG. 6 illustrates a block diagram of an exemplary embodiment of a pathology report linker of FIG. 5 in accordance with the inventive principles of the present disclosure.

FIG. 7 illustrates a block diagram of an exemplary embodiment of a pathology location label identifier of FIG. 5 in accordance with the inventive principles of the present disclosure.

FIG. 8A illustrates a block diagram of an exemplary embodiment of a biopsy sample label mapper of FIG. 5 in accordance with the inventive principles of the present disclosure.

FIG. 8B illustrates an exemplary biopsy sample label mapping in accordance with the inventive principles of the present disclosure.

FIG. 9 illustrates a block diagram of an exemplary embodiment of a biopsy sample label linker of FIG. 5 in accordance with the inventive principles of the present disclosure.

### DETAILED DESCRIPTION OF THE PREFERRED EMBODIMENTS

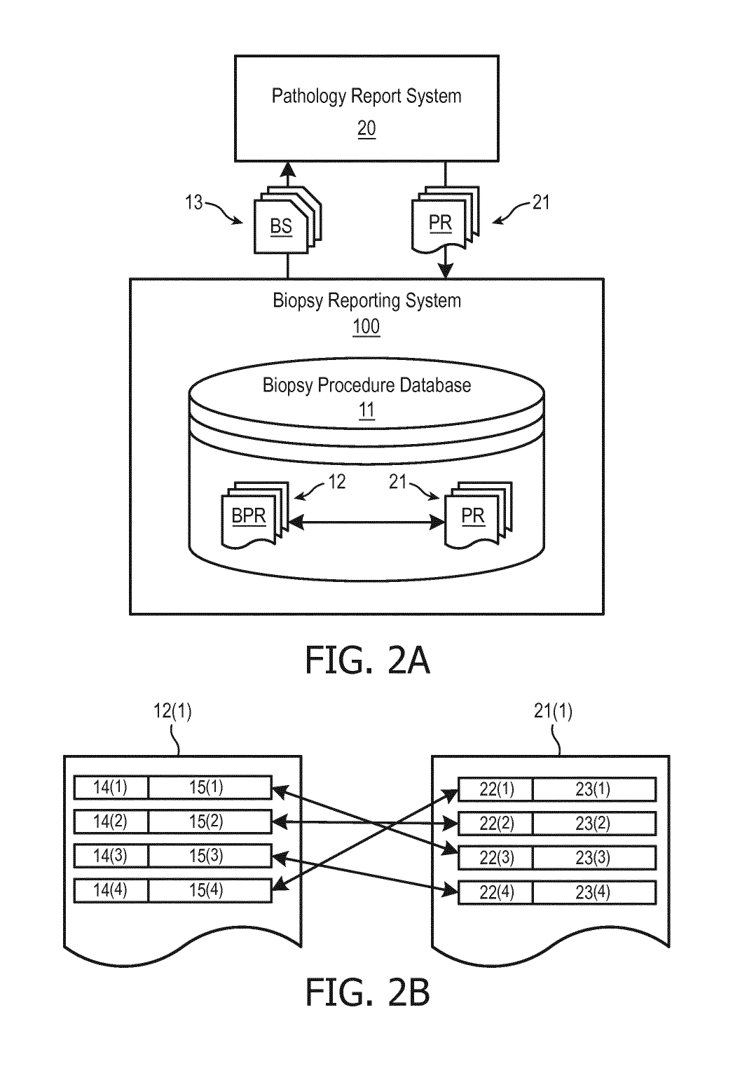

FIG. 2A illustrates a biopsy reporting system 100 of the present disclosure for executing a biopsy procedure (e.g., targeted and/or systematic) for registering extracted biopsy samples in biopsy procedure database 11 as known in the art of the present disclosure and for further providing an automatic linking, as symbolic shown, of a pathology report 21 to a respective biopsy procedure registration 12 in biopsy procedure database 11 in accordance with the inventive principles of the present disclosure.

Biopsy procedure registration 12 includes sample tissue data record informative of biopsy extractions of a plurality of biopsy samples from a patient anatomy (e.g., a prostate, a liver, etc.). More particularly, each biopsy extraction of biopsy procedure registration 12 as stored within biopsy procedure database 11 will be identified by a biopsy sample label associated with a description of the extracted sample tissue.

Pathology report 21 includes sample diagnostic data entry informative of a pathological diagnosis of each biopsy sample of the plurality of biopsy samples. More particularly, each diagnosed biopsy sample of pathology report 21 as stored within biopsy procedure database 11 will be identified by a pathology location label associated with a description of the pathology diagnosis of a biopsy sample as located within the patient anatomy. The pathology location labels of pathology report are typically derived from the biopsy location labels of containers 13, but the pathology location labels in practice are not necessarily identical to or equivalent to the biopsy location labels of containers 13.

FIG. 2B illustrates an automatically linking in accordance with the inventive principles of the present disclosure by biopsy reporting system 100, as symbolically shown, of:
1. sample tissue data record 15(1) of biopsy procedure registration 12(1) a with corresponding sample diagnostic data entry 23(3) reported in pathology report 21(1);
2. sample tissue data record 15(2) of biopsy procedure registration 12(1) with corresponding sample diagnostic data entry 23(2) reported in pathology report 21(1),
3. sample tissue data record 15(3) of biopsy procedure registration 12(1) with corresponding sample diagnostic data entry 23(4) reported in pathology report 21(1); and
4. sample tissue data record 15(4) of biopsy procedure registration 12(1) with a corresponding sample diagnostic data entry 23(1) reported in pathology report 21(1).

The aforementioned automatic linking by biopsy reporting system 100, as will be further described herein, minimally disrupts a biopsy workflow between an extracted biopsy samples transmitted within containers 13 to pathology report system 10 and a review of a pathology report 21 informative of a pathological diagnosis of the extracted biopsy samples.

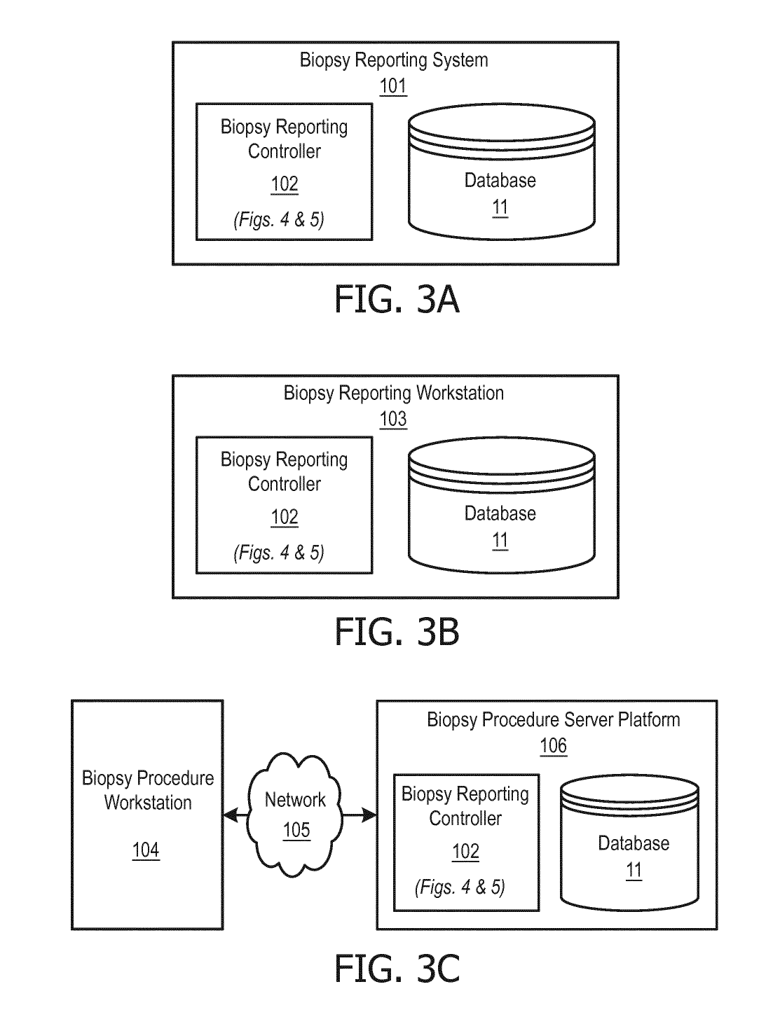

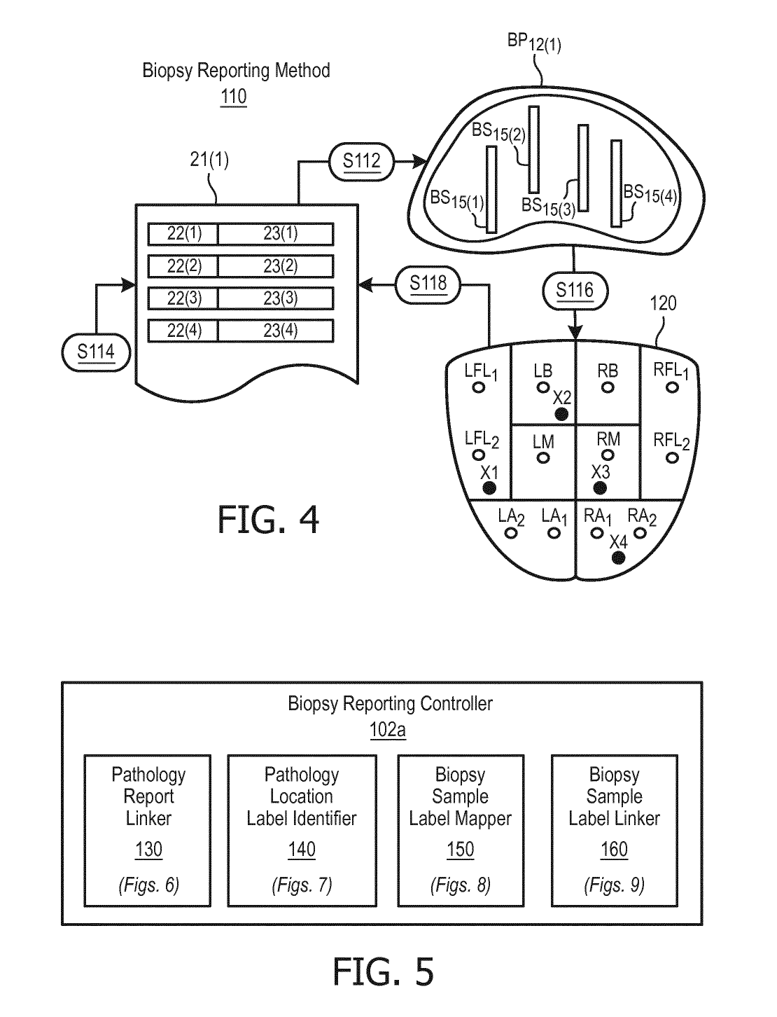

To facilitate an understanding of the inventions of the present disclosure, the following description of FIG. 3A-4 teaches basic inventive principles of an exemplary biopsy reporting controller and an exemplary biopsy reporting method of the present disclosure. From this description, those having ordinary skill in the art will appreciate how to apply the inventive principles of the present disclosure to making and using numerous and varied embodiments of biopsy reporting controllers and biopsy reporting methods of the present disclosure of the present disclosure.

Referring to FIG. 3A, a biopsy reporting system 101 of the present disclosure employs a biopsy reporting controller 102 and biopsy procedure database 11.

In practice, biopsy reporting controller 102 may be embodied in numerous and various manners within biopsy reporting system 100a.

In a first embodiment, biopsy reporting controller 102 and biopsy procedure database 11 are installed within a biopsy reporting workstation 103, which includes additional components (not shown for clarity) customarily associated with a workstation including, but not limited to, a monitor and one or more user input devices (e.g., a keyboards and a mouse). In practice, biopsy reporting workstation 103 further employs controllers, modules, etc. (not shown for clarity) for performing a targeted biopsy procedure and/or a systematic biopsy procedure and for registering the extracted biopsy samples as known in the art of the present disclosure.

In a second embodiment, biopsy reporting controller 102 and biopsy procedure database 11 are alternatively installed within a biopsy procedure server platform 106 remotely accessible by a biopsy reporting workstation 104 via a network 105 (e.g., a local area network, a virtual private network, a storage area network, etc.) or alternatively a datalink.

In practice, biopsy reporting controller 102 may employ a processor, a memory, a user interface, a network interface, and a storage interconnected via one or more system buses.

The processor may be any hardware device capable of executing instructions stored in memory or storage or otherwise processing data. As such, the processor may include a microprocessor, field programmable gate array (FPGA), application-specific integrated circuit (ASIC), or other similar devices.

The memory may include various memories such as, for example L1, L2, or L3 cache or system memory. As such, the memory may include static random access memory (SRAM), dynamic RAM (DRAM), flash memory, read only memory (ROM), or other similar memory devices.

The user interface may include one or more devices for enabling communication with a user such as an administrator. For example, the user interface may include a display, a mouse, and a keyboard for receiving user commands. In some embodiments, the user interface may include a command line interface or graphical user interface that may be presented to a remote terminal via the network interface.

The network interface may include one or more devices for enabling communication with other hardware devices. For example, the network interface may include a network interface card (NIC) configured to communicate according to the Ethernet protocol. Additionally, the network interface may implement a TCP/IP stack for communication according to the TCP/IP protocols. Various alternative or additional hardware or configurations for the network interface will be apparent.

The storage may include one or more machine-readable storage media such as read-only memory (ROM), random-access memory (RAM), magnetic disk storage media, optical storage media, flash-memory devices, or similar storage media. In various embodiments, the storage may store instructions for execution by the processor or data upon with the processor may operate. For example, the storage may store a base operating system for controlling various basic operations of the hardware.

To accomplish the automatic linking, biopsy reporting controller 102 executes a biopsy reporting method of the present disclosure. In one embodiment, biopsy reporting controller 102 executes a biopsy reporting method 110 as shown in FIG. 4.

Referring to FIG. 4, a stage S112 of method 110 encompasses biopsy reporting controller 102 linking a pathology report corresponding to a particular biopsy procedure of interest, such as, for example as shown in FIG. 4, an identification by biopsy reporting controller 102 of a pathology report 21(1) corresponding to a biopsy procedure BP₁₂₍₁₎ of four (4) extracted biopsy samples BS₁₅₍₁₎-BS₁₅₍₄₎ of a prostate as registered by biopsy procedure registration 12(1) (FIG. 2B) stored within biopsy procedure database 11.

A stage S114 of method 110 encompasses biopsy reporting controller 102 identifying pathology location labels in the linked pathology report of stage S112, such as, for example as shown in FIG. 4, an identification by biopsy reporting controller 102 of pathology location labels 22(1)-22(4) in the identified pathology report 21(1). As previously described, pathology location labels 22(1)-22(4) tag four (4) sample pathology data 23(1)-23(4) informative of the pathological diagnosis of the four (4) extracted biopsy samples BS₁₅₍₁₎-BS₁₅₍₄₎ within the prostate. However, as previously described herein, the art of the present disclosure does not link pathology location labels 22(1)-22(4) within pathology report 21 to biopsy sample labels 14(1)-14(4) (FIG. 2B) of the extracted biopsy samples BS₁₅₍₁₎-BS₁₅₍₄₎ of biopsy procedure registration 12(1) (FIG. 2B) as stored within the biopsy procedure database 11. Stages S116 and S118 of method 110 therefore provide for an automatic linkage of pathology location labels 22(1)-22(4) of pathology report 21 to biopsy sample labels 14(1)-14(4) of the extracted biopsy samples BS₁₅₍₁₎-BS₁₅₍₄₎ of biopsy procedure registration 12(1).

Specifically, stage S116 of method 110 encompasses biopsy reporting controller 102 mapping extracted biopsy samples at sample schematic locations within an anatomical schematic of the patient anatomy whereby the anatomical schematic has an area/regional nomenclature. Each sample schematic location is provided a schematic location label corresponding to the area/region associated with the sample schematic location to thereby assign a mapping within the anatomical schematic of biopsy sample labels corresponding to the sample schematic locations of the extracted biopsy samples.

For example, as shown in FIG. 4, biopsy reporting controller 102 maps each extracted biopsy samples BS₁₅₍₁₎-BS₁₅₍₄₎ at respective sample schematic locations X1-X4 with an anatomical schematic 120 of a prostate whereby anatomical schematic 120 has a regional nomenclature LFL₁, LFL₂, LB, LM, RB, RM, RFL₁, RFL₂, LA₁, LA₂, RA₁ and RA₂ as known in the art of the present disclosure. Each sample schematic location X1-X4 is provided a schematic location label corresponding to the region associated with the sample schematic location to thereby assign a mapping within anatomical schematic nomenclature 120 of biopsy sample labels 14(1)-14(4) corresponding to respective schematic location labels LFL₂(X1), LB(X2), RM(X3) and RA₂(X4) of extracted biopsy samples BS₁₅₍₁₎-BS₁₅₍₄₎.

Stage S118 of method 110 encompasses biopsy reporting controller 102 associating each identified pathological location label of stage S114 to one of the schematic location labels of the extracted biopsy samples to thereby link each identified pathological location label of stage S114 to one of the biopsy sample labels, such as, for example as shown in FIG. 4, an association by biopsy reporting controller 102 of each pathology location labels 22(1)-22(4) to one of the schematic location labels LFL₂(X1), LB(X2), RM(X3) and RA₂(X4) of extracted biopsy samples BS₁₅₍₁₎-BS₁₅₍₄₎ to thereby link each pathology location labels 22(1)-22(4) to a corresponding one of the biopsy sample labels 14(1)-14(4) (e.g., a database table linking) As a result each sample diagnostic data entry 23(1)-23(4) is now linked to a corresponding sample tissue data record 15(1)-15(4) informative of each biopsy sample tissue extraction specified in biopsy procedure registration 12(1).

In practice, the area/regional nomenclature of the anatomical schematic may be identical to or equivalent to the nomenclature of the identified pathological location label of stage S114, which facilitates a straightforward association during stage S118 of each identified pathological location label of stage S114 to one of the schematic location labels of the extracted biopsy samples. Alternatively, information within each sample diagnostic data entry may be matched to one of the corresponding registered sample tissue data records of the biopsy procedure registration as further exemplary described herein.

Also in practice, the present disclosure contemplates numerous and varied embodiments of biopsy reporting controller 102 for executing biopsy reporting method 110.

In one embodiment as shown in FIG. 5, a biopsy reporting controller 102a employs a pathology report linker 130, a pathological location label identifier 140, a biopsy sample label mapper 150 and a biopsy sample label linker 160.

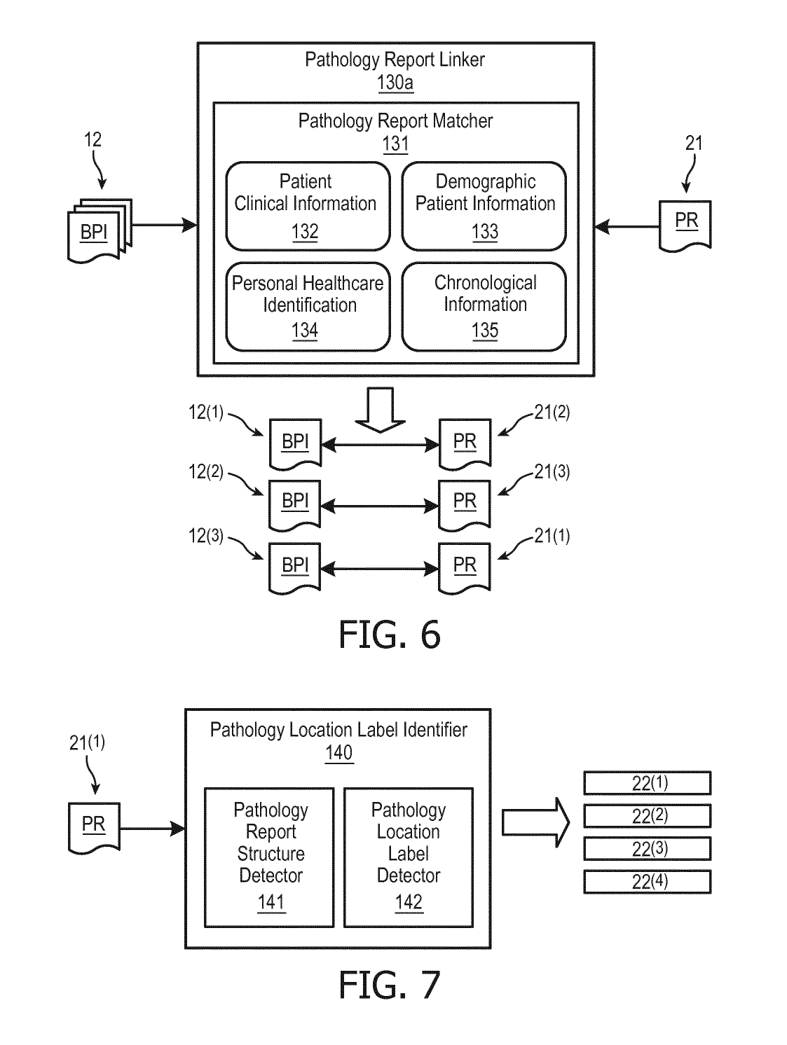

Pathology report linker 130 is an application module for linking a pathology report that corresponds to one of a plurality of biopsy procedure registrations stored within the biopsy procedure database 11 (FIG. 3A). In one embodiment as shown in FIG. 6, a pathology report linker 130a employs a pathology report matcher 131 structurally configured to establish a database table linking between the pathology report to one of a plurality of biopsy procedure registrations stored in a biopsy procedure system, such as, for example:
1. a database table linking of a pathology report 21(1) to a biopsy procedure registration 12(3);
2. a database table linking of a pathology report 21(2) to a biopsy procedure registration 12(3); and
3. a database table linking of a pathology report 21(3) to a biopsy procedure registration 12(2).

In practice, pathology report matcher 131 processes information contained within the biopsy procedure registration and the pathology report to create the biopsy procedure database table linking based on a closet informational match derived from a matching technique as known in the art of the present disclosure (e.g., a data fusion, a data merging or a synthetic matching). Examples of such information include, but are not limited to:
1. Patient clinical information 132: Clinical history, medication, and problem list associated with biopsy procedure registrations 12 and pathology reports 21;
2. Demographic patient information 133 associated with biopsy procedure registrations 12 and pathology reports 21;
3. Personal healthcare identification 134: MRN (medical record number), patient number, pathology reports number and exam procedure number; and
4. Chronological information 135: Dates associated with biopsy procedure registrations 12 and pathology reports 21.

Referring back to FIG. 5, pathology location label identifier 140 is an application module for identifying pathology location labels in the pathology report identified by pathology report linker 130. In one embodiment as shown in FIG. 7, a pathology location label identifier 140a employs a pathology report structure detector 141 and a pathology location label detector 142 to identify pathology location labels in the pathology report identified by pathology report linker 130, such as, for example, an identification of pathology location labels 22(1)-22(4) within pathology report 21(1)

(FIG. 4).

In practice, pathology report structure detector 141 is structurally configured to identify section(s), paragraph(s) and sentence(s) of the pathology report by a structural implementation of a Natural Language Processing (“NLP”) and/or heuristic rules as known in the art of the present disclosure.

In practice, pathology location label detector 142 is structurally configured to process the identified section(s), paragraph(s) and sentence(s) of the pathology report by biopsy report structure detector 141 to thereby identify the pathology location labels by a structural implementation of a NLP, Regular Expression (“Regex”), medical ontologies (e.g., SNOMED and RadLex) and/or other label identification techniques as known in the art of the present disclosure.

Referring back to FIG. 5, biopsy sample label mapper 150 is an application module for mapping extracted biopsy samples of a biopsy procedure registration to an anatomical schematic having a regional nomenclature whereby the anatomical schematic has an area/regional nomenclature. Each sample schematic location is provided a schematic location label corresponding to the region associated with the sample schematic location to thereby assign a mapping within the anatomical schematic of biopsy sample labels corresponding to the sample schematic locations of the extracted biopsy samples.

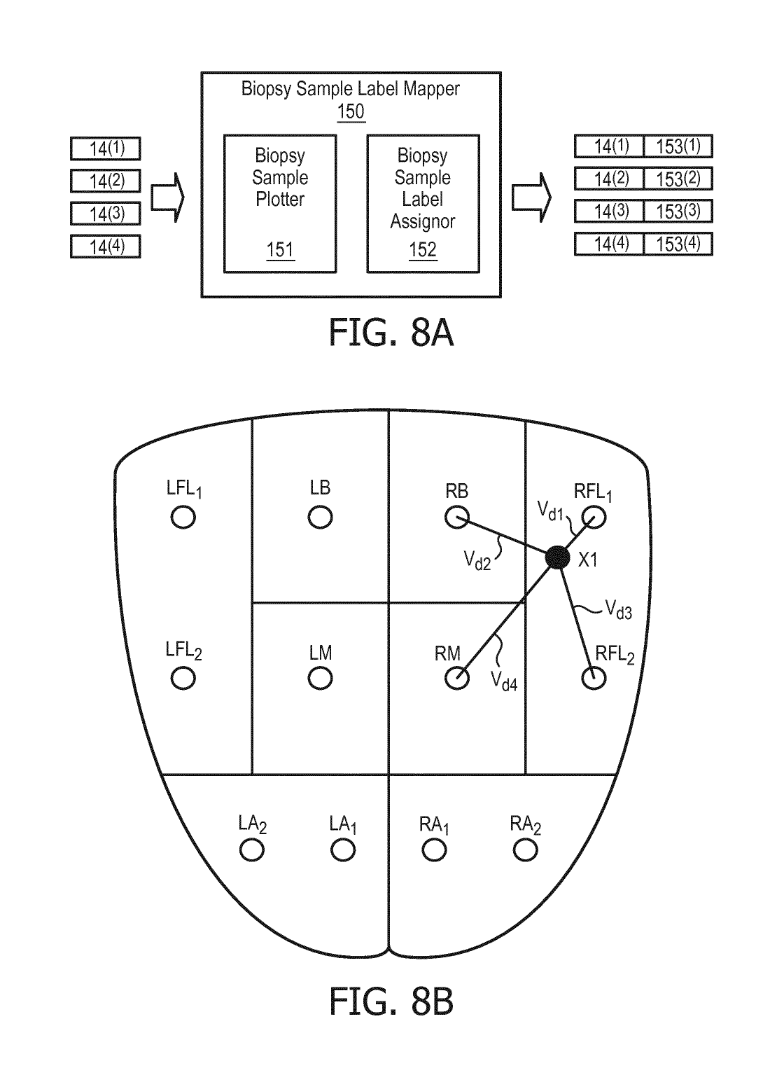

In one embodiment as shown in FIG. 8, a biopsy sample label mapper 150 employs a biopsy sample plotter 151 and a biopsy sample label assignor 152 for mapping extracted biopsy samples of a biopsy procedure registration to an anatomical schematic having a regional nomenclature to thereby assign biopsy sample labels corresponding to respective sample schematic locations of the extracted biopsy samples, such as, for example, a mapping of extracted biopsy samples BS₁₅₍₁₎-BS₁₅₍₄₎ (FIG. 4) at respective sample schematic locations X1-X4 with an anatomical schematic 120 of a prostate (FIG. 4) to thereby assign biopsy sample labels 14(1)-14(4) corresponding to respective sample schematic location labels LFL₂(X1), LB(X2), RM(X3) and RA₂(X4) of extracted biopsy samples BS₁₅₍₁₎-BS₁₅₍₄₎.

In practice, biopsy sample plotter 151 is structurally configured to translate the extracted biopsy samples in a biopsy procedure registration (e.g., extracted biopsy samples BS₁₅₍₁₎-BS₁₅₍₄₎ of biopsy procedure registration 12(1) (FIG. 4)) into an anatomical schematic of a regional nomenclature (e.g., anatomy schematic 120 of a prostate having a regional nomenclature) by structurally implementing a utilization of a location where the biopsy samples were collected as indicated by the associated sample tissue data record 15(1)-15(4).

For example, to illustrate the approach by biopsy sample plotter 151, R is a set of prostate schematic diagram regions {r₁, r₂, . . . , r_{M}} and L is a set of schematic based labels {l₁, l₂, . . . , l_{N}}. For the prostate schematic diagram of FIG. 8B, R=8 regions and L=12 labels including:
1. Labels LFL₁ and LFL₂ in a region 1;
2. Label LB in a region 2;
3. Label LM in a region 3;
4. Label RB in a region 4;
5. Label RM in a region 5;
6. Labels RFL₁ and RFL₂ in region 6;
7. Labels LA₁ and LA₂ in region 7; and
8. Labels RA₁ and RA₂ in region 8;

Biopsy sample plotter 151 plots a centroid of each label within a corresponding region in accordance with h(L,R), which is a function providing a centroid of a label lᵢ in a region rⱼ. Centroids plotted by biopsy sample plotter 151 for this example are represented by the white circles underneath the labels of FIG. 8B.

Biopsy sample plotter 151 further plots the extracted biopsy samples in accordance with set locations {x₁, x₂, . . . , x_{P}}, where the extracted biopsy samples were removed in a biopsy procedure and P≤N. For example, a set location Xl as shown in FIG. 8B represents a plotted location of a biopsy sample BS₁₅₍₁₎ (FIG. 4). In practice, biopsy sample label assignor 152 is structurally configured to assign a schematic location label 153 to each plotted location x₁ by finding minimal distance between a plotted location x₁ and all centroids of labels L. For example, as shown in FIG. 8B:
1. a vector V_{d1} represents a distance between a centroid of label RFL₁ and plotted location X1;
2. a vector V_{d2} represents a distance between a centroid of label RB and plotted location X1;
3. a vector V_{d3} represents a distance between a centroid of label RFL₂ and plotted location X1; and
4. a vector V_{d4} represents a distance between a centroid of label RM and plotted location X1.

In view of vector V_{d1} being the minimal distance vector, biopsy sample labeler 152 assigns schematic location label 153(1) of RFL₁ to plotted location X1, which corresponds to biopsy sample BS₁₅₍₁₎ and associated biopsy sample label 14(1). Upon a completion of the plotting of all of the extracted biopsy samples within the anatomical schematic and the assignment of schematic location labels to each plotted location, then each schematic location label will be associated with a biopsy sample label of a corresponding extracted biopsy sample.

Referring back to FIG. 5, biopsy sample label linker 160 is an application module for linking a pathology location label identified by pathology location label identifier 140 within the pathology report linked to the biopsy procedure registration by pathology report linker 130 to a biopsy sample label assigned by biopsy sample label mapper 150 associated with a schematic location label.

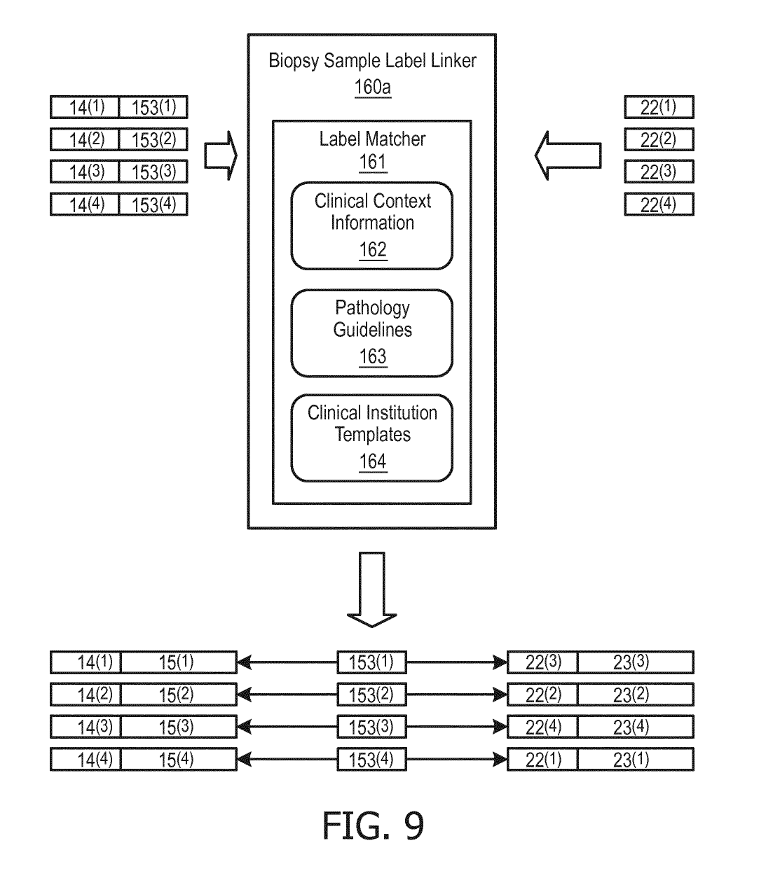

In one embodiment as shown in FIG. 9, a biopsy sample label linker 160a employs a label matcher 161 structurally configured to match a pathology location label identified within the pathology report linked to the biopsy procedure registration to a biopsy sample label assigned by biopsy sample label mapper 150 associated with a schematic location label, such as, for example:
1. a database table linking of a pathology location label 22(1) within a pathology report 21(1) (FIG. 2) to a biopsy sample label 14(4) of a biopsy procedure registration 12(1) (FIG. 2) via schematic location label 153(4);
2. a database table linking of a pathology location label 22(2) within pathology report 21(1) to a biopsy sample label 14(2) of biopsy procedure registration 12(1) via schematic location label 153(2);
3. a database table linking of a pathology location label 22(3) within pathology report 21(1) to a biopsy sample label 14(1) of biopsy procedure registration 12(1) via schematic location label 153(1); and
4. a database table linking of a pathology location label 22(4) within pathology report 21(1) to a biopsy sample label 14(3) of biopsy procedure registration 12(1) via schematic location label 153(3).

In practice, biopsy label matcher 161 implements an informational matching technique as known in the art of the present disclosure based on information relevant to the biopsy procedure that facilitates a matching of a sample diagnostic data entry of pathology record to a sample tissue data record of a linked biopsy procedure registration as facilitated by the schematic location label. Examples of such information include, but are not limited to:
1. clinical context information (e.g., family cancer history, smoking history and chemotherapy treatment);
2. pathology guidelines (e.g., Protocol for the Examination of Specimens From Patients With Carcinoma of the Prostate Gland from College of American Pathologist and the Guidelines on processing and reporting of prostate biopsies from the European Randomized Study of Screening for Prostate Cancer Committee); and
3. clinical institution templates of specific guidelines to comply with clinical and quality principles.

Further in practice, biopsy label matcher 161 may provide for a validation of match of a sample diagnostic data entry of pathology record to a sample tissue data record of a linked biopsy procedure registration via a user interface.

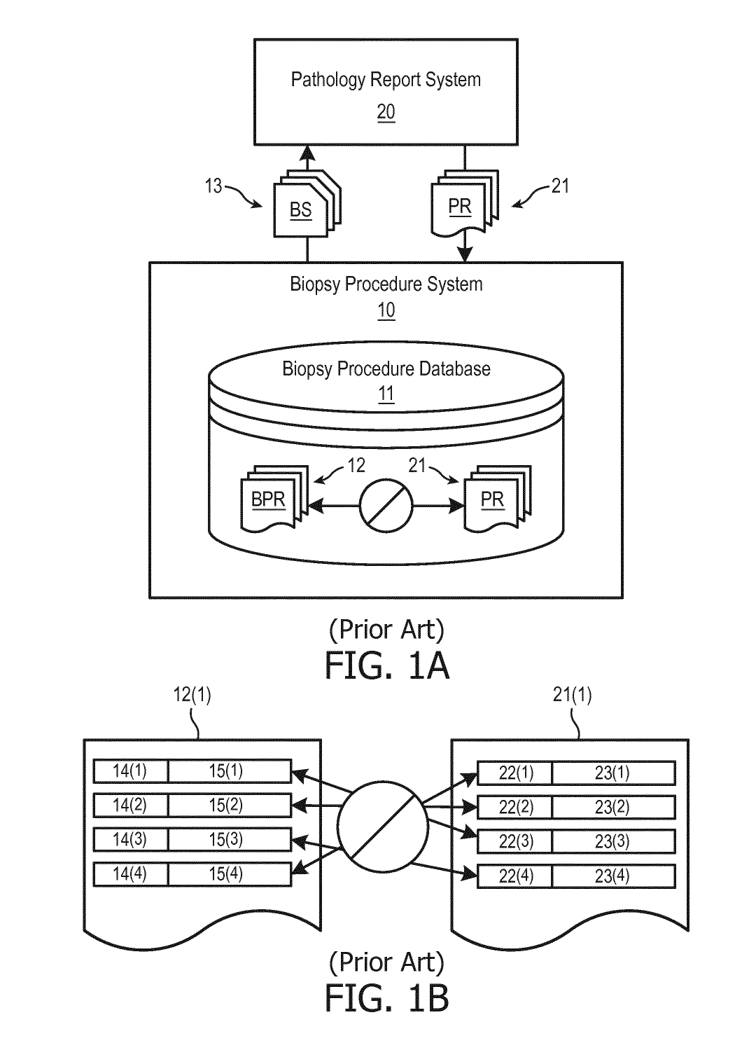

Referring to FIGS. 1-9, those having ordinary skill in the art of the present disclosure will appreciate numerous benefits of the inventions of the present disclosure including, but not limited to, (1) a fully integrated environment enabling an easy navigation between a specific pathological diagnosis of a sample tissue and their respective location in the biopsy system, and (2) a minimizing of human introduced errors caused by the manual approach of linking information of biopsy and pathology.

Furthermore, as one having ordinary skill in the art will appreciate in view of the teachings provided herein, features, elements, components, etc. described in the present disclosure/specification and/or depicted in the Figures may be implemented in various combinations of electronic components/circuitry, hardware, executable software and executable firmware and provide functions which may be combined in a single element or multiple elements. For example, the functions of the various features, elements, components, etc. shown/illustrated/depicted in the Figures can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, circuitry, combinations thereof, etc.) which is capable of (and/or configurable) to perform and/or control a process.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar function, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Furthermore, exemplary embodiments of the present disclosure can take the form of a computer program product or application module accessible from a computer-usable and/or computer-readable storage medium providing program code and/or instructions for use by or in connection with, e.g., a computer or any instruction execution system. In accordance with the present disclosure, a computer-usable or computer readable storage medium can be any apparatus that can, e.g., include, store, communicate, propagate or transport the program for use by or in connection with the instruction execution system, apparatus or device. Such exemplary medium can be, e.g., an electronic, magnetic, optical, electromagnetic, infrared or semiconductor system (or apparatus or device) or a propagation medium. Examples of a computer-readable medium include, e.g., a semiconductor or solid state memory, magnetic tape, a removable computer diskette, a random access memory (RAM), a read-only memory (ROM), flash (drive), a rigid magnetic disk and an optical disk. Current examples of optical disks include compact disk read only memory (CD-ROM), compact disk read/write (CD-R/W) and DVD. Further, it should be understood that any new computer-readable medium which may hereafter be developed should also be considered as computer-readable medium as may be used or referred to in accordance with exemplary embodiments of the present disclosure and disclosure.

Having described preferred and exemplary embodiments of novel and inventive anatomical models for position planning and tool guidance during a medical procedure (which embodiments are intended to be illustrative and not limiting), it is noted that modifications and variations can be made by persons having ordinary skill in the art in light of the teachings provided herein, including the Figures. It is therefore to be understood that changes can be made in/to the preferred and exemplary embodiments of the present disclosure which are within the scope of the embodiments disclosed herein.

Moreover, it is contemplated that corresponding and/or related systems incorporating and/or implementing the device or such as may be used/implemented in a device in accordance with the present disclosure are also contemplated and considered to be within the scope of the present disclosure. Further, corresponding and/or related method for manufacturing and/or using a device and/or system in accordance with the present disclosure are also contemplated and considered to be within the scope of the present disclosure.

1. A biopsy reporting system, comprising:
a biopsy procedure database operable to store a biopsy procedure registration and a pathology report,
wherein the biopsy procedure registration includes sample tissue data informative of biopsy extractions of a plurality of biopsy samples from a patient anatomy, and
wherein the pathology report includes sample diagnostic data informative of a pathological diagnosis of each biopsy sample of the plurality of biopsy samples; and
a biopsy reporting controller,
wherein, responsive to a storage of the biopsy procedure registration and the pathology report within the biopsy procedure database, the biopsy reporting controller is structurally configured to automatically link the pathology report to the biopsy procedure registration, and
wherein, responsive to an automatic linking by the biopsy reporting controller of the pathology report to the biopsy procedure registration, the biopsy reporting controller is structurally configured to automatically link a pathological diagnosis of each biopsy sample to a corresponding biopsy extraction of each biopsy sample.

2. The biopsy reporting system of claim 1, further comprising:
a biopsy reporting workstation,
wherein the biopsy reporting controller and the biopsy procedure database are installed with the biopsy reporting workstation, or
wherein the biopsy reporting controller and the biopsy procedure database are remotely accessible by the biopsy reporting workstation.

3. (canceled)

4. The biopsy reporting system claim 1,
wherein the biopsy reporting controller includes a pathology report linker structurally configured to link a pathology report as stored within the biopsy procedure database to a corresponding biopsy procedure registration among a plurality of biopsy procedure registrations stored within the biopsy procedure database.

5. The biopsy reporting system of claim 1,
wherein the pathology report linker includes a pathology report matcher structurally configured to match the pathology report to the biopsy procedure registration based at least one of patient clinical information, demographic patient information, personal healthcare information and chronological information.

6. The biopsy reporting system of claim 4,
wherein the biopsy reporting controller includes a pathology location label identifier structurally configured to identity a plurality of pathology location labels within the pathology report; and
wherein each pathology location label serves as an identifier of one of a plurality of sample diagnostic data entries within the pathology report.

7. The biopsy reporting system of claim 6, wherein the pathology location label identifier includes a pathology report structure detector structurally configured to identify at least one of sections, paragraphs and sentences of the pathology report, and a pathology location label detector structurally configured to detect the pathology locution labels within the pathology report based on identification by the pathology report structure detector of the at least one of sections, paragraphs and sentences of the pathology report.

8. (canceled)

9. The biopsy reporting system of claim 6,
wherein the biopsy reporting controller includes a biopsy sample label mapper structurally configured to map a plurality of biopsy sample labels to an anatomical schematic; and
wherein each biopsy sample label serves as an identifier of one of a plurality of sample tissue data records of the biopsy procedure registration.

10. The biopsy reporting system of claim 9,
wherein the biopsy sample label mapper includes a biopsy sample plotter structurally configured to plot the biopsy samples of the corresponding biopsy procedure registration into the anatomical schematic.

11. The biopsy reporting system of claim 10,
wherein the biopsy sample label mapper includes a biopsy sample label assignor structurally configured to assign each biopsy sample label among the plurality of biopsy sample labels to a corresponding biopsy sample of the corresponding biopsy procedure registration plotted into the anatomical schematic.

12. The biopsy reporting system of claim 11,
wherein an assignment by the biopsy sample label assignor of each biopsy sample label among the plurality of biopsy sample labels to a corresponding biopsy sample is derived from distance vectors extending from a plotting by the biopsy sample map plotter of the biopsy samples of the biopsy procedure registration to labeled centroid locations in the anatomical schematic.

13. The biopsy reporting system of claim 9,
wherein the biopsy reporting controller includes a biopsy sample label linker structurally configured to automatically link each biopsy sample label among the plurality of biopsy sample labels to a corresponding pathology location label among the plurality of pathology location labels responsive to an identification by the pathology location label identifier of the pathological location labels within the pathology report and to a mapping by the biopsy sample label mapper of the biopsy sample labels within the biopsy procedure registration to the anatomical schematic; and
wherein the biopsy sample label linker includes a label matcher structurally configured to match each biopsy sample label among the plurality of biopsy sample labels to a corresponding pathology location label among the plurality of pathology location labels based on at least one of clinical context information, pathology guidelines and clinical institution templates.

14. (canceled)

15. A biopsy reporting controller, comprising:
a pathology report linker structurally configured to automatically link a pathology report stored within a biopsy procedure database to a corresponding biopsy procedure registration among a plurality of biopsy procedure registrations stored within the biopsy procedure database,
wherein the biopsy procedure registration includes sample tissue data informative of biopsy extractions of a plurality of biopsy samples from a patient anatomy, and
wherein the pathology report includes sample diagnostic data informative of a pathological diagnosis of each biopsy sample of the plurality of biopsy samples.
a pathology location label identifier structurally configured to identify pathology location labels within the pathology report responsive to a linkage by the pathology report linker of the pathology report to the corresponding biopsy procedure registration,
wherein each pathology location label serves as an identifier of one of a plurality of sample diagnostic data entries within the pathology report;
a biopsy sample label mapper structurally configured to map a plurality of biopsy sample labels of the biopsy procedure registration to an anatomical schematic using distance vectors extending from a plotting of the biopsy samples of the biopsy procedure registration to labeled centroid locations in the anatomical schematic,
wherein each biopsy sample label serves as an identifier of one of a plurality of sample tissue data records of the biopsy procedure registration; and
a biopsy sample label linker structurally configured to automatically link each biology sample label among the plurality of biology sample labels to a corresponding pathology location label among the plurality of pathology location responsive to an identification by the pathology location label identifier of the pathology location labels within the pathology report and further responsive to a mapping by the biopsy sample label mapper of the biopsy sample labels to the anatomical schematic.

16. A biopsy reporting method, comprising:
storing a biopsy procedure registration and a pathology report within a biopsy procedure database,
wherein the biopsy procedure registration includes sample tissue data informative of biopsy extractions of a plurality of biopsy samples from a patient anatomy, and
wherein the pathology report includes sample diagnostic data informative of a pathological diagnosis of each biopsy sample of the plurality of biopsy samples;
automatically linking the pathology report to the biopsy procedure registration by a biopsy reporting controller responsive to the storing of the biopsy procedure registration and the pathology report within the biopsy procedure database; and
automatically linking a pathological diagnosis of each biopsy sample to a corresponding biopsy extraction of each biopsy sample by the biopsy reporting controlling responsive to the automatic linking by the biopsy reporting controller of the pathology report to the biopsy procedure registration.

17. The biopsy reporting method of claim 16, wherein the automatic linking by the biopsy reporting controller of the pathology report to the biopsy procedure registration includes:
automatically linking an informational correspondence of the pathology report to the biopsy procedure registration among a plurality of biopsy procedure registrations stored within the biopsy procedure database.

18. The biopsy reporting method of claim 16, wherein the automatically linking by the biopsy reporting controller of the pathological diagnosis of each biopsy sample to the corresponding biopsy extraction of each biopsy sample includes:
identifying pathology location labels with the pathology report,
wherein each pathology location label serves as an identifier of one of a plurality of sample diagnostic data entries within the pathology report:
assigning a plurality of biopsy sample labels of the biopsy procedure registration to a mapping of the biopsy samples of the biopsy procedure registration into an anatomical schematic,
wherein each biopsy sample label serves as an identifier of one of a plurality of sample tissue data records of the biopsy procedure registration; and
automatically linking each biopsy sample labels among the plurality of biopsy sample labels to a corresponding pathology location label among the plurality of pathology location labels based on the identifying of the pathology location labels with the pathology report and assigning a plurality of biopsy sample labels of the biopsy procedure registration to the mapping of the biopsy samples of the biopsy procedure registration into the anatomical schematic.

19. (canceled)

20. (canceled)
